(19) United States
(12) Patent Application Publication
(10) Pub. No.: US 2003/0059819 A1
(43) Pub. Date: Mar. 27, 2003

(54) Combination of epoxy and amine silanes for immobilizing and hybridizing nucleic acid molecules on a solid support

(75) Inventors: Chi-Meng Tzeng, Taipei (TW); Kung-Ming Hsu, Taipei (TW); Wei-Chi Ku, Taoyuan (TW); Grace Lau, Taipei (TW); Jing-Tyan Ma, Taipei (TW); Sung-Kay Chiu, Taipei (TW)

Correspondence Address: COHEN, PONTANI, LIEBERMAN & PAVANE, Suite 1210, 551 Fifth Avenue, New York, NY, 10176, US

(73) Assignee: U-Vision Biotech, Inc.

(21) Appl. No.: 10/226,787

(22) Filed: Aug. 23, 2002

Related U.S. Application Data
(60) Provisional application No. 60/314,936, filed on Aug. 24, 2001.

(51) Int. Cl. C12Q 1/68; C12M 1/34; B05D 3/00

(52) U.S. Cl. 435/6; 435/287.2; 427/2.11

(57) ABSTRACT

A composition for coating a solid support to immobilize and hybridize nucleic acid molecules on the solid support comprises an amine silane and an epoxy silane. A nucleic acid microarray and a method of manufacturing such microarray using such composition are described.

### BRIEF DESCRIPTION OF THE DRAWINGS

In the drawings:

FIG. 1 shows a synergistic effect of two types of silanes on immobilization and hybridization of unmodified or amine-linked DNA, where (A) demonstrates the signals of Cy3-labeled G3PDH PCR product (983 bp) immobilized on slides coated with epoxysilane, aminesilane, and epoxy+aminesilane; and (B) demonstrates the signals from hybridization of the G3PDH PCR product with the labeled cDNA of the same gene. In both A and B, 1 represents the signals from the epoxysilane slide, 2, from aminesilane slide; 3, summation of the signals of epoxysilane (1) and amine silane (2) slides together; 4, signals from epoxy+aminesilane mixture slides.

FIG. 2 shows optimization of the hybridization of G3PDH PCR product (983 bp) by changing the concentration of one silane at a time on the mixture-silane slide surface, where (A) depicts the effect of variation of aminesilane while keeping the concentration of epoxysilane at 1% and (B) the effect of variation of epoxysilane while the concentration of aminesilane was kept at 1%.

FIG. 3 shows the effects of the two types of silanes on hybridization of nucleic acids at a high humidity or by UV irradiation. Signals from hybridization of the nucleic acids with their respectively labeled complementary DNAs were measured on the slides that had been treated by either one of the two immobilization methods after the DNAs were printed on the slides, where oligonucleotides (70mer and 30mer) (A) or long PCR products (2322 bp and 983 bp) (B) were kept either in a humid chamber or in the drawer (humidity at 65 to 70%) for different lengths of time (from 0 to 24 hours). Alternatively, oligonucleotides (10 to 70mer) (C) or long PCR products (983, 556, and 298 bp) (D) were spotted and irradiated with UV light at different dosages indicated. (E) is a direct comparison of the signals from both methods.

FIG. 4 illustrates stronger immobilization of DNA on the slide and reusability of the slide, where (A) shows the signals from 30 spots of Cy5 labeled G3PDH PCR product (983 bp) immediate after printed on the slide (S), the signals in (A) from the slides being measured after the slides were washed with 0.1%SDS twice for 5 min and then boiled for 2 min (WI) and the signals after another round of the same treatment (W2), and (B) shows that an unlabeled 800 bp PCR product and a 70mer oligonucleotide were spotted on the slides and hybridized with a Cy3 labeled complementary 70mer overnight, the signals being obtained from the hybridization (Hyb1) and after stripping (Strip 1). The same procedure was repeated two more rounds, and the signals after hybridization and after stripping were Hyb2, Hyb3 and Strip2, Strip3, respectively.

FIG. 5 is a comparison of immobilization of long PCR products on different slide surfaces, where (A) depicts the efficiency of immobilization that was calculated as the fraction of the labeled PCR product (983 bp long) that retained on the slide surface after treated with the whole hybridization procedure except without hybridizing with a labeled DNA, amine+epoxy representing the mixture slide developed in this paper, amine representing a slide from TeleChem, 3-D polymer representing a slide from SurModics, and poly-L-lysine representing a slide that was made in-house according to the protocol from Brown's laboratory, and (B) shows the images and the values of the DNA spots before and after washing. In (B), the signal mean and the signal/noise ratio are the values from 18 spots and the variations of signals are shown as a standard deviation (st dev); the signal-to-noise ratio is calculated by dividing the signal means with the background means; the rainbow color bar represents the signal intensity of the spots from scanning; and the red color stands for the highest signal intensity and purple color the lowest intensity.

FIG. 6 demonstrates maximization of hybridization signals from oligonucleotide on slide surface by diluting the concentrations of both coating silanes on slide surface, where (A) shows the signals of the oligonucleotides of 30, 50, and 70 nucleotides long, and (B) shows the signals from slides that were printed with PCR products of different lengths. This experiment was to show the difference of in behavior between PCR products and oligonucleotides on slide surface with different concentrations of the two silanes.

FIG. 7 is a comparative study of the hybridization capabilities of oligonucleotide and PCR product on different types of slides, where (A) shows the signals from the oligonucleotide spots after hybridization with its Cy3 labeled complementary oligonucleotide, and (B) shows a PCR product of 983 bp long was spotted and hybridized with a Cy3 labeled cDNA. The inserts are the images from scanning the slides and only three representive spots of a total 15 spots were shown. Amine-1 is Coming CMT-GAPS™ slide and Amine-2 is TeleChem SuperAmine™ slide.

### DETAILED DESCRIPTION OF THE PRESENTLY PREFERRED EMBODIMENTS

As used hereinafter, the term “solid support” refers to any suitable materials with solid surfaces, which include, but are not limited to, glass, polymers solid phase, plastics, mica, alumina (Al₂O₃), titania (TiO₂), SnO₂, RuO₂, PtO₂, as well as other metal oxide surfaces.

The term “hybridizable element” means a biomolecule that has the ability to hybridize with its complementary structure or sequence, such as a single strand DNA, an RNA and a PNA. Methods of performing hybridization reactions are commonly known to a person of ordinary skill in the art and also are described by, for example, Sambrook, J. el al., Molecular Cloning: A Laboratory Manual, Cold Spring Hoarbor Laboratory Press, Cold Spring Harbor, N.Y. (1989); Haymes, B. D. et al., Nucleic Acid Hybridization: A Practical Approach, IRL Press, Washington, D.C. (1985); and Keller, G. H. and Manak, M. M., DNA Probes, Second Edition, Stockton Press, New York, N.Y. (1993). All these references are hereby incorporated by reference.

The term “interactable element” means a biomolecule that is capable of specifically binding to another biomolecule or a chemical compound, such as a protein.

The term “substrate” means a solid support that is treated with chemicals such that it is capable of binding or being associated with biomolecules, either covalently or non-covalently.

The term “nucleic acid molecule” includes all DNA, RNA, PCR products, oligonucleotides, whether naturally occurring or synthetic, single stranded or double stranded, and modified or unmodified.

The efficiency of different compounds, such as amine, epoxy, or aldehyde silanes to immobilize nucleic acids on glass slides surface is different. However, the effect of a combination of two different types of compounds, such as a combination of an amine silane and an epoxy silane, or amine silane and an aldehyde silane, or an epoxy silane and an aldehyde silane, was unknown prior to the present invention.

Synergistic Effect of a Combination of Silanes on Nucleic Acid Binding

To investigate the effect of the combination of silanes on the efficiency of immobilizing nucleic acids on a solid surface such as a glass slide, the solid surface is coated either with a single silane or a mixture of two silanes of three types: aminesilane, epoxysilane, and aldehyde-silane. Fluorescent dye-labeled PCR products were then spotted and immobilized onto slide surfaces coated with different types of salines, and the slides were then treated with washing buffers that were used in hybridization. This treatment was to test the percentage of DNA that could be leaked out from the slide surface. The fluorescent signals remained on the slide surface after spotting and treatment with various washing buffers were measured. The amount or percentage of PCR products remained on the slide surface varied tremendously depending on the types of silanes.

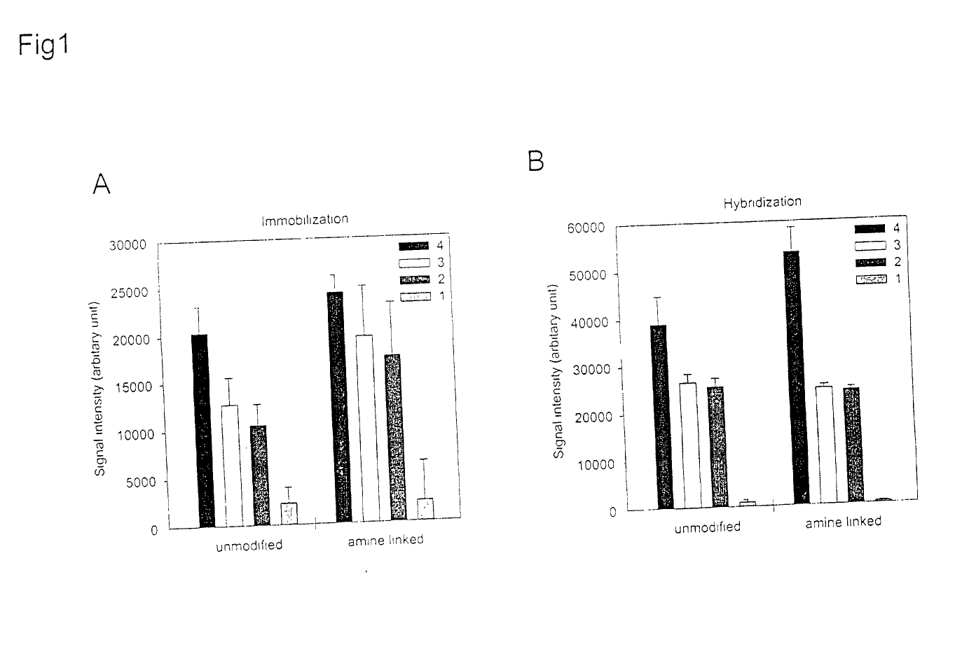

As shown in FIG. 1A, the amount of DNA immobilized on an epoxysilane slide (light gray bar) was much less than that on an amine slide (dark gray bar). Interestingly, the binding ability of the epoxy+amine mixture slide (black bar) was twice as much as that of aminesilane slide instead of just a little above that of aminesilane slide from the addition of the signals of the two slides (white bar). The same effect was observed when the spotted PCR products were amino-modified (right-hand panel of FIG. 1A). The results of the experiments suggest that there is a synergistic effect between the epoxy group and amine group on the binding of DNA onto the slide surface. The strong interaction from the positively charged tertiary amine of the silane molecule and the negatively charged phosphate-backbone of the DNA helps the nucleic acids to be immobilized longer on the slide surface, whilst the epoxy group can form a covalent linkage with the primary amine groups on the bases of the DNA. The presence of both interactions enhances each other to create the synergistic effect. It is also contemplated in the present invention that the combination of an amine silane and an aldehyde silane or an aldehyde silane and an epoxy silane may result in similar synergistic effects.

We further investigated whether the immobilized DNA was available for hybridization by testing with labeled PCR probe (FIG. 1B). The DNA on the epoxysilane slides could yield only very low signal (light gray bar) compared with that on the aminesilane slide (dark gray bar). The signals of hybridization from the epoxy+amine mixture slides (black bar) were around twice as much as those on the aminesilane slide when the DNA was amine-modified. However, binding of the unmodified DNA on an mixture slide was only 50% better than on an amine-alone slide (left-hand panel of FIG. 1B). Similar results had also been obtained when different concentrations of DNA were spotted. These observations suggest that the additional immobilized DNA on the epoxy+amine mixture slide is highly available for hybridization.

Optimization of the Ratio of the Two Types of Silanes

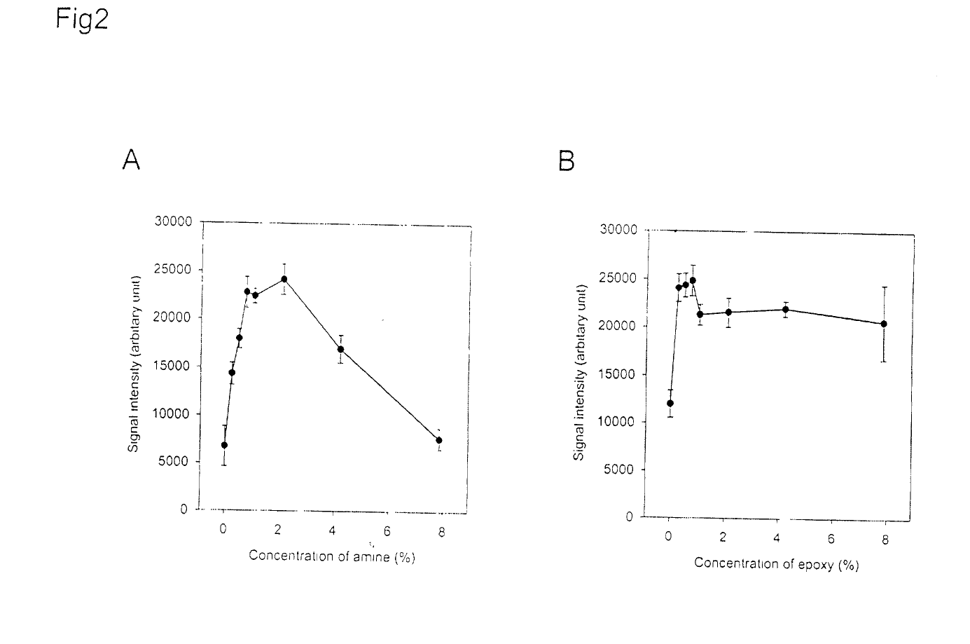

We further determined the optimal concentration (percentage composition) of the two silanes to obtain the highest signal of hybridization from immobilized PCR products by varying the concentration of one silane while keeping the other one constant. As depicted in FIG. 2A, increasing the concentration of just aminesilane enhanced the signal of hybridization very sharply and reached the maximum at around 1.0 to 2.0% and then dropped linearly. The experiments suggest that changing the ratio of the two silanes from 1:1 leads to reduction of the interaction between the DNA and the slide surface.

On the other hand, when the concentration of epoxy-silane was increased while keeping aminesilane at 1%, the signals of hybridization increased sharply but reached a plateau around 0.5% (FIG. 2B). Unlike aminesilane, when epoxy concentration was higher than 1.0%, the hybridization signals did not increase or decrease further (FIG. 2B). Thus, we kept the concentrations of epoxy-silane and aminesilane at 1.0% for most of the other experiments because it is the optimal concentration for yielding the highest hybridization signals. In many other experiments we showed that the DNA immobilization seemed to be maximal when the ratio of the two silanes is around 1:1.

These two sets of experiment demonstrated that the proportion or ratio between the two types of silanes is important in creating the synergistic effect that helps to immobilize DNA on the slide surface.

Comparison of Immobilization Methods

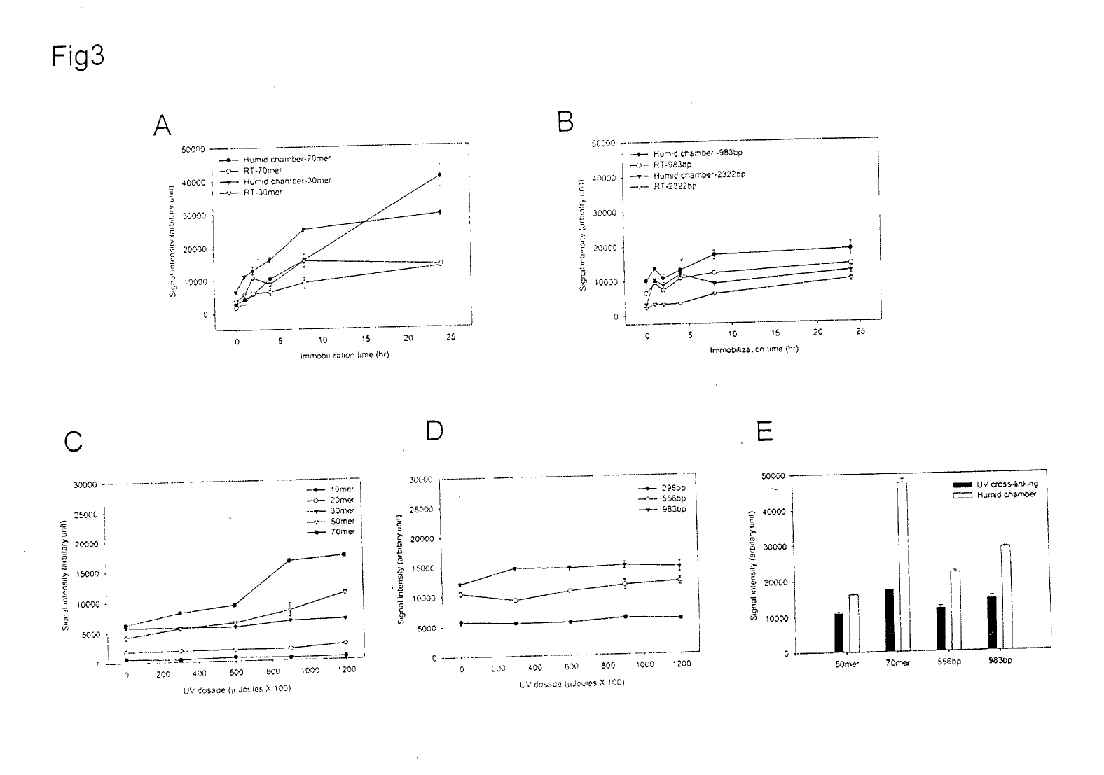

Since the epoxy group reacts with the amine side-chain more favorably in the presence of water, therefore, we kept the slides in a humid chamber for different length of time at 42° C. to observe the change in efficiency of immobilization of nucleic acids. As shown in FIG. 3A, the amounts of oligonucleotide (of different lengths) immobilized on the slide surfaces are increased with the length of time under the humid condition (filled circles). Such effect seemed to be enhanced even after 24 hours. The enhancement by high humidity was less obvious when the slides were put in a laboratory drawer where the humidity was around 65-70% (open circles). Humidity only had a slight effect on the immobilization of long PCR products, for example, from 983 to 2300 bps., and it was not as dramatic as that on oligonucleotides (FIG. 3B).

The other major method to immobilize nucleic acids on a slide surface is to form a covalent linkage between the nucleic acids and the slide by UV irradiation. Thus, we investigated the effect of different dosages of ultraviolet light on nucleic acid immobilization. There were only slight increases in the binding of nucleic acids on the slide surface when the dosage of UV light was increased (FIG. 3C and D). Moreover, the slight increase seemed to be more effective when the oligonucleotides were longer (70mer vs. 30 mer). The effect of UV dosage on long PCR products was almost negligible in the range of UV dosage applied (FIG. 3D).

Thus, between the two methods of immobilization, i.e. immobilization by humidity or UV irradiation, the immobilization method at 42° C. in a humid chamber was preferred because the humidity methods is more effectiveness and simple without requiring an additional instrument (UV crosslinker) (FIG. 3E).

Stronger Binding of Nucleic Acids on the Solid Surface

The experiments described above suggest that the nucleic acids could be bound tightly on a solid surface, therefore, we went on to investigate whether the nucleic acids bound on the slide could survive harsh treatments.

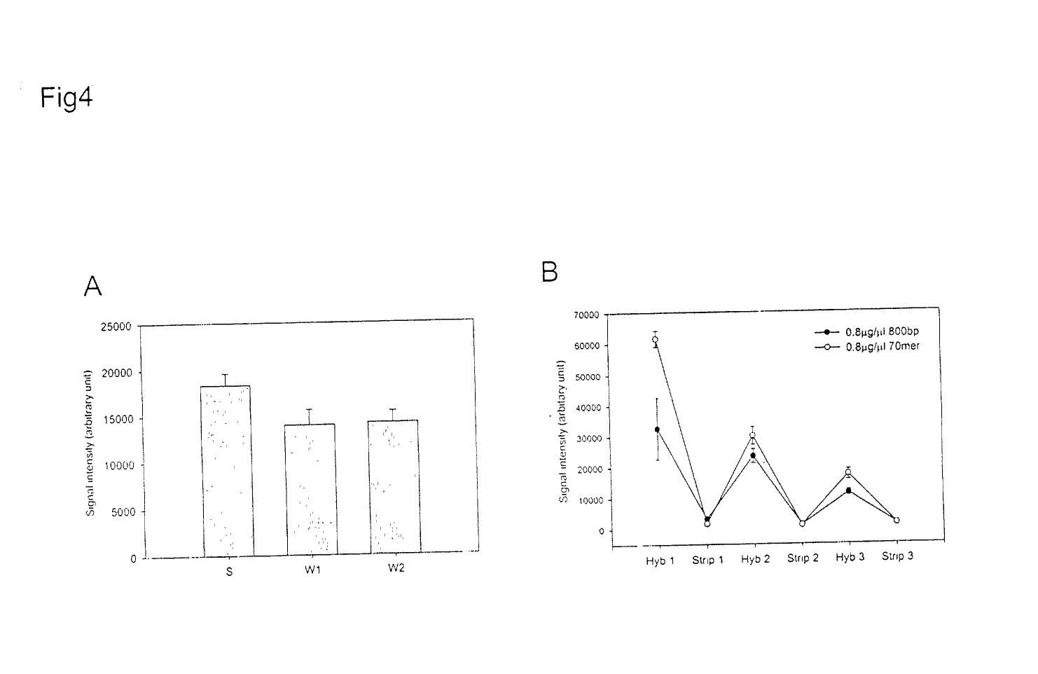

The first set of harsh treatment was after spotting and immobilization of a Cy3-labeled PCR product of 1 k bp on a slide surface, the slide were then washed with 0.1%SDS twice and boiled for 2 min to remove any unbound DNA from the slide. Such conditions are similar to the conditions for de-hybridizing or stripping the complementary sequences from the oligonucleotides or the PCR products bound on the solid surfaces. After the first wash and boiling, there were 76.5% of the DNA bound on the surface compared with that before the treatment (FIG. 4A). Then the slide was treated a second time under the same de-hybridization (stripping) condition and the remaining DNAs was not further washed away, suggesting that the bound nucleic acids were linked to the slide very tightly. The result of the experiment led us to believe that the slides coated with the combination of salines can survive the harsh treatment, such as stripping, so that such slides may be reuseable for hybridization purposes.

In the second set of experiment, we spotted and immobilized an 800 bp PCR product and a 70mer oligonucleotide on slide surfaces. After hybridization with a Cy3-labeled 70mer oligonucleotide complementary to both DNAs, hybridization signals (Hyb) were obtained by scanning (FIG. 4B) the slide. The signals were then stripped by boiling in a stripping buffer for 10 min, and the slides were scanned again (Strip). The above procedure was repeated two more times. As shown in FIG. 4B, there was a gradual decrease of signals (70% to 60% loss on average) after each round of hybridization and stripping. The decrease in the ability of the DNA to be hybridized after stripping may be explained by two possibilities. One possibility is that more bound DNA on the slide surface may be removed by the harsh stripping treatment which was much more stringent than that of the last experiment by washing with SDS and boiling in water for 2 min. The proportion of oligonucleotide removed by each round of stripping was more than that of the long PCR product because oligonucleotide molecules lack the “spagetti” effect a tangling of the long denatured DNA strand helps to be trapped inside the mass on the slide surface. The second possibility is that the PCR product on the slide surface might be less available due to the change in the layer structure of the DNA on the slide surface (14). The results demonstrates that the slides were reusable because majority of the DNAs retained on the slides were still available for the next hybridization and the residual background signals after stripping (6 to 10%) were still well below the coefficient of variation of the signals if the slides were used for repeating the similar experiments.

Comparison of the Binding Efficiency Among Compounds

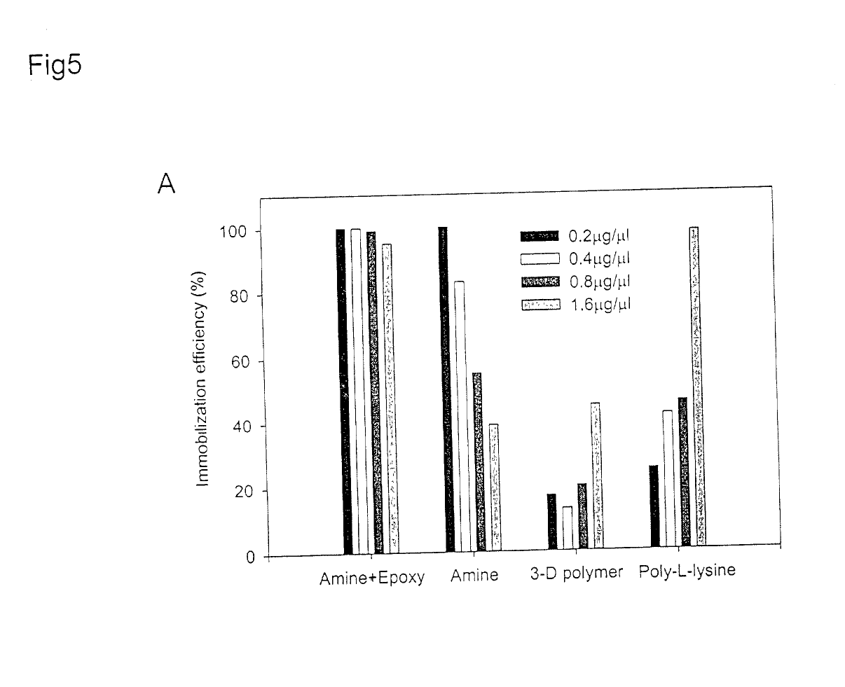

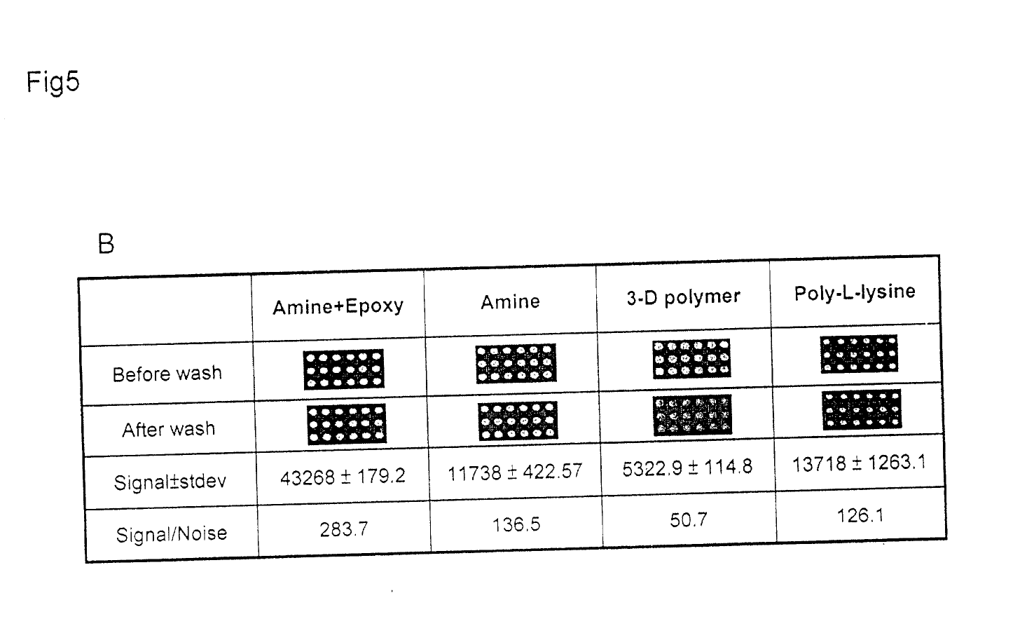

We compared the DNA immobilization efficiency of the solid surfaces coated with different compounds. Slides coated with amine +epoxy silanes (mixture slide), amine saline alone, poly-L-lysine, or polymeric substrates were used for the comparison. The efficiencies of DNA binding on different slides varied greatly shown by spotting with a Cy-labeled PCR product (FIG. 5A). With the amine+epoxy-silane slides, the DNA binding seemed to be very efficient and was almost independent of the concentration of the DNA spotted (from 0.2 to 1.6 μg/μl). The other types of slide had different efficiencies of binding depending on the concentration of the DNA. For example, the aminesilane slide could bind most of the DNA when the concentration was low, while around half of the DNA could be washed away when the concentration of the DNA was high. Surprisingly, the behavior of poly-L-lysine-coated slide was exactly the opposite. Higher DNA concentration resulted in more DNA binding. We believe that it may be due to the facts that different surfaces with different hydrophobicities would allow different amounts of DNA to be transferred (FIG. 5B, signal before wash), and the sizes of the spots are also different. Moreover, the actual amount of DNA bound to the slides (after wash) and the signal-to-noise ratios were highest on the mixture slide, while the other slides had lower signals and signal-to-noise ratios (FIG. 5B).

Thus, the combination of salines provide a better substrate for DNA immobilization and hybridization on a solid surface.

Optimization of Oligonucleotide Binding

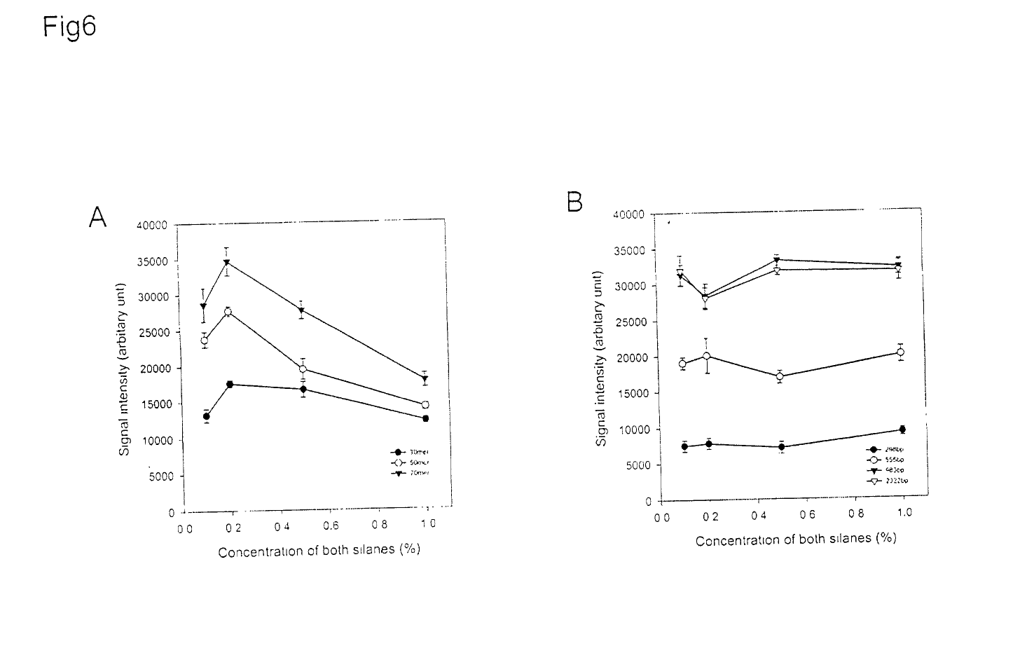

During the optimization process we observed that the efficiency of binding of oligonucleotide might also depend on the concentration of silane on the slide surface. Therefore, we determined the concentrations of both epoxy and amine silanes that could lead to the maximal binding and hybridization of oligonucleotides of different lengths. At 0.2% of both epoxy and amine silanes were the concentrations that oligonucleotides from 30 to 70 mers hybridized with the highest efficiency (FIG. 6A). However, a similar trend in hybridization with PCR products on the slide surface was not observed. Instead, the signals seemed to be independent of the concentration of both silanes (FIG. 6B).

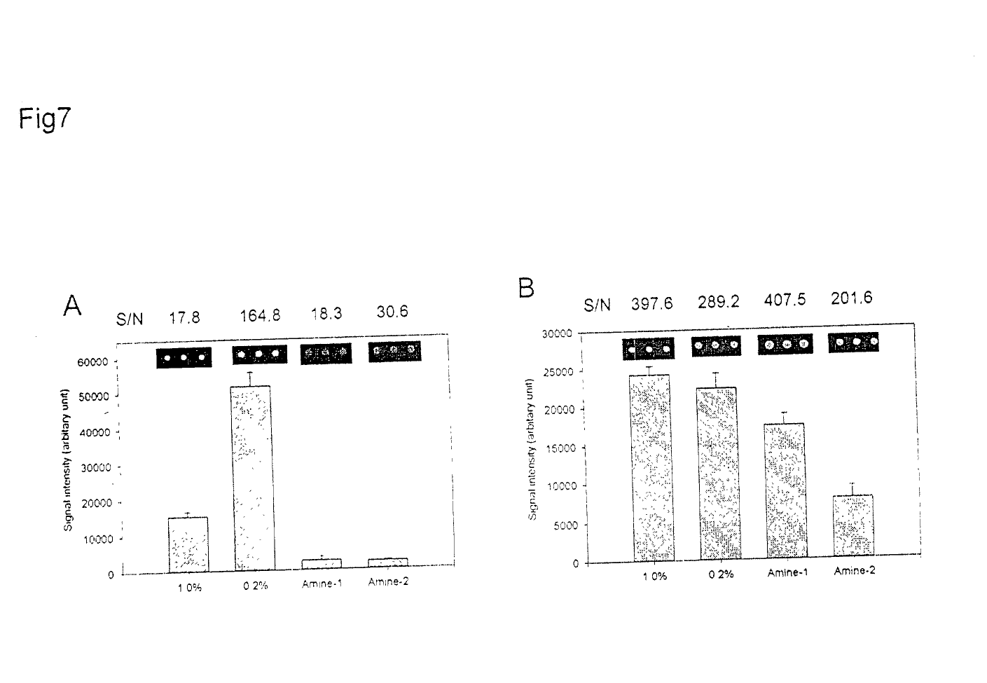

Finally, we carried out a comparative study to find out the differences among other compounds in hybridization efficiency of oligonucleotides and long DNA molecules. As shown in FIG. 7A, the decrease in the concentration (0.2%) of the mixture silanes resulted in 3-fold increase in the signal by comparing with the original formula (1%) that described above in connection with the PCR product immobilization. Moreover, the capacity and thus hybridization efficiency of oligonucleotide on the slide were much higher than those of other amine silane coated slides (FIG. 7A, amine-1 and amine-2). The 0.2% mixture silane slide was also the one with the highest signal-to-noise ratio (S/N=164.8) while the S/N ratios of other slides were around 20 to 30. When an 800 bp PCR product was spotted on the 0.2% mixture saline slides, it did not show much difference in hybridization signals (FIG. 7B) compared with those on the 1% mizture silane slide. It appears that a lower concentration of the mixture silanes allows oligonucleotide to bind more efficiently.

In sum, the present application describes a method to increase the binding capacity and efficiency of hybridization of nucleic acids onto a solid surface such as a glass slide, using two types of silanes such as an amine silane and an epoxy silane, having different binding mechanisms, one being ionic and the other covalent. The binding capability of the solid surface coated with mixture of the silanes is much stronger than any single silane-coated slides such that the immobilized DNA can survive very harsh washing and stripping treatments. Also, lowering of the concentrations of both silanes at the same time allows much higher efficiency of hybridization of the oligonucleotides on the slide surface. The resulting slide surface has a very high capacity and thus high signal-to-noise ratio compared with the surfaces coated with other compounds.

Tetraethylorthosillicate may be added to the coating solution that comprises two type of silanes, as a spacer, to control the density of the silanes and the interaction with the silicate glass surface.

It will be readily apparent to one skilled in the art that various substitutions and modifications may be made to the invention disclosed herein without departing from the scope and spirit of the present invention.

The following references are provided to further describe and illustrate the present invention. The contents of these references are hereby incorporated by reference in their entirety.

### REFERENCES

1. Ramsay, G. (1998) DNA chips: state-of-the art. Nat. Biotechnol., 16, 40-44.

2. Fodor, S. P. A., Read, J. L., Pirrung, M. C., Stryer, L., Lu, A. T., and Solas, D. (1991) Light-directed, spatially addressable parallel chemical synthesis. Science, 251, 767-773.

3. Singh-Gasson, S., Green, R. D., Yue, Y., Nelson, C., Blattner, F., Sussman, M. R., and Cerrina, F. (1999) Maskless fabrication of light-directed oligonucleotide microarrays usinga digital micromirror array. Nature Biotechnol., 17, 974-978.

4. Cohen, G., Deutsch, J., Fineberg, J., and Levine, A. (1997) Covalent attachment of DNA oligonucleotides to glass. Nucleic Acids Res, 25, 911-912.

5. Kumar, A., Larsson, O., Parodi, D., and Liang, Z. (2000) Silanized nucleic acids: a general platform for DNA immobilization. Nucleic Acids Res., 28, e71.

6. Okamoto, T., Suzuki, T., and Yamamoto, N. (2000) Microarray fabrication with covalent attachment of DNA using bubble jet technology. Nat. Biotechnol., 18, 384-385.

7. Rogers, Y. H., Jiang-Baucom, P., Huang, Z. J., Bogdanov, V., Anderson, S., and Boyce-Jacino, M. T. (1999) Immobilization of oligonucleotides onto a glass support via disulfide bonds: A method for preparation of DNA microarrays. Anal. Biochem., 266, 23-30.

8. Schena, M., Shalon, D., Davis, R. W., and Brown, P. O. (1995) Quantitative monitoring of gene expression patterns with a complementary DNA microarray. Science, 270, 476-470.

9. Bowtell, D. D. L. (1999) Options available-from start to finish-for obtaining expression data by microarray. Nature Genet., 21 (suppl.), 25-32.

10. Zammatteo, N., Jeanmart, L., Hamels, S., Courtois, S., Loutte, P., Hevesi, L., and Remacle, J. (2000) Comparison between different strategies of covalent attachment of DNA to glass surfaces to build DNA microarrays. Anal. Biochem., 280, 143-150.

11. Lee, P. H., Sawan, S. P., Modrusan, Z., Arnold, L. J., and Reynolds, M. A. (2002) An efficient binding chemistry for glass polynucleotide microarrays. Bioconjug. Chem., 13, 97-103.

12. Dolan, P. L., Wu, Y., Ista, L. K., Metzenberg, R. L., Nelson, M. A., and Lopez, G. P. (2001) Robust and efficient synthetic method for forming DNA microarrays. Nucleic Acids Res., 29, e107.

13. Beier, M., and Hoheisel, J. D. (1999) Versatile derivatisation of solid support media for covalent bonding on DNA-microchips. Nucleic Acids Res., 27, 1970-1977.

14. Steel, A. B., Levicky, R. L., Heme, T. M., and Tarlov, M. J. (2000) Immobilization of nucleic acids at solid surfaces: Effect of oligonucleotide length on layer assembly. Biophysical Journal, 79, 975-981.

The following examples represent some particular embodiments of the present invention, which shall not be construed as limitations of various aspects of the present invention.

Materials and Reagents Used in the Examples

DMSO, sodium borohydride, sodium chloride, sodium citrate, sodium dodecyl sulphate, ethanol, formamide were all from Merck (NJ, USA). Poly(A)n was bought from MWG-Biotech Inc. (Ebersberg, Germany) and human Cot-1 was from Invitrogen (Carlsbad, Calif., USA). Unmodified, amine-linked, and Cy5, Cy3-labeled oligonucleotides were synthesized by MWG-Biotech Inc. The sequences of the oligonucleotide were designed by an in-house computer program, U-GET (U-Vision Biotech, Taiwan), to be unique and have the minimal degree of secondary structure. Aminesilane slides were purchased from TeleChem International Inc. (CA, USA) and Coming Inc. (NY, USA). 3D polymer microarray slides were from SurModics (MN, USA).

Apparent to a person of ordinary skill in the art, the above listed materials and reagents or their equivalents may also be obtained from any other sources and readily substituted by a person of ordinary skill in the art without substantially altering the results of the present invention. Other materials mentioned in the present application, which are not listed above, are also readily obtainable.

### EXAMPLE 1

### Preparation of the Coating Solution

For every 100 ml of coating solution, the following components were mixed in a 50 ml tube: 0.1 ml tetraethylorthosilicate, 0.21 ml (3-glycidyloxypropy) trimethoxysilane, 0.21 ml (N,N-diethyl-3-aminopropyl) trimethoxysilane, 0.12 tetramethylammonium hydroxide and 1.36 ml of 100% alcohol. The mixture was then stirred at room temperature for two hours. 46 ml of 100% alcohol was added to the mixture, followed by a 10 min stirring. After such solution was then divided equally into two tubes, 26 ml of 100% alcohol were added to each tube to make a total of 100 ml coating solution., respectively. Each coating solution was then filtered with 0.45 μm filter an wrapped with aluminum foil for storage. Such coating solution has a shelf life of about one month at room temperature.

### EXAMPLE 2

### Preparation of Substrate (Coated Slides)

The super white slides (75.3×25.2×1.1 mm, Innotest) were cleaned and placed in the chamber of a Swienco type 40 PM 40 spin coater (SOP 0050). A 200 μl coating solution of the Example 1 above was applied to the middle of each slide to allow the solution to spread out on the surface. The slides were then spun at 6,000 rpm for 12 seconds. The coated slides were then transferred to an oven and baked at 80° C. for 20-24 hours. The slides can be stored at room temperature for about 12 months.

### EXAMPLE 3

### Fabrication of Microarray Slides

The coating solution was made by stirring a mixture of 0.1 to 1% of (3-glycidyloxypropyl)trimethoxysilane (UCT, PA, USA) and 0.1 to 1% of (N,N-diethyl-3-aminopropyl)trimethoxysilane (UCT) and tetramethylammonium hydroxide (Lancaster, Morecambe, England) at 4° C. for 30 min. Glass slides were cleaned by ultrasonication in double-distilled water for 30 min, soaked in 10% NaOH for 60 min, rinsed with running water for 5 min, and then dried by spinning at 50×g for 5 min. The slides were then coated with a monolayer of coating solution by immersing the slides in the coating solution for 5 min before spinning in a spin coater. The polymerization of the silanes on the slide surface was then accelerated by incubating at 80° C. for 20 hrs.

Printing of Cy3 or Cy5-labeled or unlabeled PCR products or oligonucleotides was performed by using a Cartesian PixSys 5500 Arrayer (Irvine, USA). The concentrations of the printed nucleic acids were from 0.2 to 0.5 μg/μl for PCR products and 0.5 to 1.2 μg/μl for oligonucleotides. After spotting the nucleic acids were immobilized by placing in a humid chamber, made by putting a small volume of saturated sodium chloride solution on the bottom of a beaker, at 42° C. for 2 hours for PCR products and 20 hrs for oligonucleotides. After immobilization the slides were then washed with vigorous agitation in 0.1% SDS for 1 min at room temperature and washed further gently in the solution for 5 min. After that the slides were immersed in boiling water for 2 min. The slides were then rinsed with ddH₂O and dried by spinning at 50×g for 5 min before using for printing. The slides were then stored at room temperature before using for hybridization.

### EXAMPLE 4

### Preparation of RNA Transcripts

Total RNA was isolated using TRIZOL reagent (Invitrogen, Carlsbad, Calif., USA), and the mRNA fraction was purified using Qiagen mRNA Midi Kit (Qiagen, Hilden, Germany). mRNA was reverse transcribed into cDNA using 13 μM of random hexamer with 1×first-strand buffer, 10 mM DTT, 500 μM of dNTP (dATP, dCTP, and dGTP), 200 μM of dTTP, 0.2U of RNasin (Invitrogen), and 13U of Superscript II (Invitrogen). 100 μM of Cy3 or Cy5-dUTP (Amersham Pharmacia Biotech, NJ, USA) was incorporated into cDNA synthesis during reverse transcription. The reaction mixture was heated to 70° C. for 10 min and placed at room temperature for 10 min to allow primer annealing. After adding Cy3 or Cy5-dUTP and enzymes, cDNA synthesis was continued at 42° C. for 90 min, followed by the addition of 30 mM sodium hydroxide at 70° C. for 15 min to hydrolyze the RNA in the mixture. The alkaline was then neutralized by adding in 30 mM hydrochloric acid. The unincorporated nucleotides were removed by using Qiagen QIAquick™ Nucleotide Removal Kit (Qiagen Inc., CA, USA) and the DNA was then concentrated by using Microcon YM-30 (Millipore Corporation, Bedford, Mass., USA). Alternatively, the mixture was purified by precipitating the solution with 70% ethanol for 1 hr.

### EXAMPLE 5

### Sample Preparation, Hybridization, Scanning, and Analysis

After the labeled cDNA derived from 1 or 2 μg of mRNA was mixed with 20 μg of Poly(dA)n and 20 μg of human Cot-1 DNA, the cDNA mixture was then concentrated with a Microcon YM-30 Concentrator (Millipore). Equal volume of 2×hybridization buffer (50% formamide, 10×SSC, and 0.2% SDS) was added in before heating at 95° C. for 3 minutes to denature the probe. The cDNA mixture was applied onto the arrays, and covered with glass cover slip. The arrays were then placed in a hybridization chamber, which was made by adding some wet towels on the bottom of a slide box to prevent the slides from drying during hybridization. The chamber was then put inside a hybridization oven at 42° C. for 16 hours when the hybridization buffer mentioned above was used, or alternatively, for one hour when EasyHyb Hybridization buffer was used (U-Vision Biotech Inc., Taiwan). The results from using either solution were very similar except the time for hybridization was different. After hybridization, the arrays were washed with 2×SSC, 0.1% SDS at 42° C. for 5 min, and 0.1×SSC, 0.1% SDS at room temperature for 10 min, and finally 0.1×SSC for 5 min. The arrays were rinsed with ddH₂O and dried by centrifugation at 50×g for 5 min. In all the experiments in the paper, the average signal values were from the spots of 3 slides processed in parallel.

Detection of fluorescent signals was performed with a ScanArray 3000 unit (Packard, USA), and the same power and PMT setting values were used for the same set of experiment. The quantification of data was performed with the Imagene 4.0 software (Biodiscovery, Inc.).

### EXAMPLE 6

### Stripping the Arrays

The fluorescent signals on the arrays were removed by boiling in a stripping solution containing 0.05×SSC, 10 mM EDTA pH 8.0, 0.1% SDS for 10 min. The slides were then rinsed with 0.01×SSC at room temperature before scanning for signal. The stripped slides were then hybridized again as the first time with the same labeled cDNA mixture. The stripping and hybridization steps were repeated two more times and the signals on the slides were collected after each round of hybridization and stripping.

We claim:

1. A composition for coating a solid support so as to immobilize and hybridize nucleic acid molecules, comprising an amine silane and an epoxy silane.

2. The composition of claim 1, wherein said amine silane is (N,N-diethyl-3-aminopropyl) trimethoxysilane.

3. The composition of claim 1, wherein said epoxy silane is (3-glycidyloxypropyl) trimethoxysilane.

4. The composition of claim 1, wherein said amine silane is (N,N-diethyl-3-aminopropyl) trimethoxysilane and said epoxy silane is (3-glycidyloxypropyl) trimethoxysilane.

5. The composition of claim 4, wherein the concentration of said (N,N-diethyl-3-aminopropyl) trimethoxysilane ranges from about 0.01% to about 8% and the concentration of said (3-glycidyloxypropyl) trimethoxysilane ranges from about 0.01% to about 8%.

6. The composition of claim 5, wherein the concentration of said (N,N-diethyl-3-aminopropyl) trimethoxysilane ranges from about 0.3% to about 4% and the concentration of said (3-glycidyloxypropyl) trimethoxysilane ranges from about 0.2% to about 4%.

7. The composition of claim 6, wherein the concentration of said (N,N-diethyl-3-aminopropyl) trimethoxysilane is about 1% and the concentration of said (3-glycidyloxypropyl) trimethoxysilane is about 1%.

8. The composition of claim 4, wherein the ratio of said (N,N-diethyl-3-aminopropyl) trimethoxysilane to said (3-glycidyloxypropyl) trimethoxysilane is about 1:1.

9. The composition of claim 4, wherein the concentration of the combination of said (N,N-diethyl-3-aminopropyl) trimethoxysilane and said (3-glycidyloxypropyl) trimethoxysilane ranges from about 0.1% to about 1%.

10. The composition of claim 9, wherein the concentration of the combination of said (N,N-diethyl-3-aminopropyl) trimethoxysilane and said (3-glycidyloxypropyl) trimethoxysilane ranges from about 0.15% to about 0.5%.

11. The composition of claim 10, wherein the concentration of the combination of said (N,N-diethyl-3-aminopropyl) trimethoxysilane and said (3-glycidyloxypropyl) trimethoxysilane is about 0.2%.

12. The composition of claim 1, further comprising tetraethylorthosilicate.

13. The composition of claim 1, wherein said solid support comprises glasses, plastics and polymers.

14. A substrate for immobilization and hybridization of nucleic acid molecules, comprising:
a. a solid support having a surface; and
b. a composition coated on said surface of said solid support to immobilize and hybridize said nucleic acid molecules, said composition comprising an amine silane and an epoxy silane.

15. The substrate of claim 14, wherein said amine silane is (N,N-diethyl-3-aminopropyl) trimethoxysilane.

16. The substrate of claim 14, wherein said epoxy silane is (3-glycidyloxypropyl) trimethoxysilane.

17. The substrate of claim 14, wherein said amine silane is (N,N-diethyl-3-aminopropyl) trimethoxysilane and said epoxy silane is (3-glycidyloxypropyl) trimethoxysilane.

18. The substrate of claim 17, wherein the concentration of said (N,N-diethyl-3-aminopropyl) trimethoxysilane ranges from about 0.01% to about 8% and the concentration of said (3-glycidyloxypropyl) trimethoxysilane ranges from about 0.01% to about 8%.

19. The substrate of claim 18, wherein the concentration of said (N,N-diethyl-3-aminopropyl) trimethoxysilane ranges from about 0.3% to about 4% and the concentration of said (3-glycidyloxypropyl) trimethoxysilane ranges from about 0.2% to about 4%.

20. The substrate of claim 19, wherein the concentration of said (N,N-diethyl-3-aminopropyl) trimethoxysilane is about 1% and the concentration of said (3-glycidyloxypropyl) trimethoxysilane is about 1%.

21. The substrate of claim 17, wherein the ratio of said (N,N-diethyl-3-aminopropyl) trimethoxysilane to said (3-glycidyloxypropyl) trimethoxysilane is about 1:1.

22. The substrate of claim 17, wherein the concentration of the combination of said (N,N-diethyl-3-aminopropyl) trimethoxysilane and said (3-glycidyloxypropyl) trimethoxysilane ranges from about 0.1% to about 1%.

23. The substrate of claim 22, wherein the concentration of the combination of said (N,N-diethyl-3-aminopropyl) trimethoxysilane and said (3-glycidyloxypropyl) trimethoxysilane ranges from about 0.15% to about 0.5%.

24. The substrate of claim 23, wherein the concentration of the combination of said (N,N-diethyl-3-aminopropyl) trimethoxysilane and said (3-glycidyloxypropyl) trimethoxysilane is about 0.2%.

25. The substrate of claim 14, further comprising tetraethylorthosilicate.

26. The substrate of claim 14, wherein said solid support comprises glasses, plastics and polymers.

27. A nucleic acid microarray, comprising:
a. a solid support;
b. a composition coated on said surface of said solid support, said composition comprising an amine silane and an epoxy silane; and
c. nucleic acid molecules immobilized on said surface of said coated solid support.

28. The microarray of claim 27, wherein said amine silane of said composition is (N,N-diethyl-3-aminopropyl) trimethoxysilane.

29. The microarray of claim 27, wherein said epoxy silane of said compostion is (3-glycidyloxypropyl) trimethoxysilane.

30. The microarray of claim 27, wherein said amine silane of said composition is (N,N-diethyl-3-aminopropyl) trimethoxysilane and said epoxy silane of said composition is (3-glycidyloxypropyl) trimethoxysilane.

31. The microarray of claim 30, wherein the concentration of said (N,N-diethyl-3-aminopropyl) trimethoxysilane ranges from about 0.01% to about 8% and the concentration of said (3-glycidyloxypropyl) trimethoxysilane ranges from about 0.01% to about 8%.

32. The microarray of claim 31, wherein the concentration of said (N,N-diethyl-3-aminopropyl) trimethoxysilane ranges from about 0.3% to about 4% and the concentration of said (3-glycidyloxypropyl) trimethoxysilane ranges from about 0.2% to about 4%.

33. The microarray of claim 32, wherein the concentration of said (N,N-diethyl-3-aminopropyl) trimethoxysilane is about 1% and the concentration of said (3-glycidyloxypropyl) trimethoxysilane is about 1%.

34. The microarray of claim 30, wherein the ratio of said (N,N-diethyl-3-aminopropyl) trimethoxysilane to said (3-glycidyloxypropyl) trimethoxysilane is about 1:1.

35. The microarray of claim 30, wherein the concentration of the combination of said (N,N-diethyl-3-aminopropyl) trimethoxysilane and said (3-glycidyloxypropyl) trimethoxysilane ranges from about 0.1% to about 1%.

36. The microarray of claim 35, wherein the concentration of the combination of said (N,N-diethyl-3-aminopropyl) trimethoxysilane and said (3-glycidyloxypropyl) trimethoxysilane ranges from about 0.15% to about 0.5%.

37. The microarray of claim 36, wherein the concentration of the combination of said (N,N-diethyl-3-aminopropyl) trimethoxysilane and said (3-glycidyloxypropyl) trimethoxysilane is about 0.2%.

38. The microarray of claim 27, further comprising tetraethylorthosilicate.

39. The microarray of claim 27, wherein said solid support comprises glasses, plastics and polymers.

40. The microarray of claim 27, wherein said nucleic acid molecules are oligo-nucleotides.

41. The microarray of claim 40, wherein said oligo-nucleotides range from 10-mer to 70-mer.

42. The microarray of claim 41, wherein said oligo-nucleotides range from 30-mer to 70-mer.

43. The microarray of claim 27, wherein said nucleic acid molecules are PCR products.

44. The microarray of claim 43, wherein said PCR products range from about 250 base pairs (bps) to about 2400 base pairs (bps).

45. The microarray of claim 44, wherein said PCR products range from about 980 bps to about 2300 bps.

46. A method of manufacturing a substrate for immobilization and hybridization of nucleic acid molecules, comprising the steps of:
a. immersing a solid support in a coating solution comprising an amine silane and an epoxy silane; and
b. coating said coated solid support by spinning in a spin coater to make said substrate.

47. The method of claim 46, further comprising the step of incubating said substrate at an elevated temperature.

48. The method of claim 47, wherein said substrate is incubated at 80° C. for 20 hours.

49. The method of claim 46, wherein said amine silane of said composition is (N,N-diethyl-3-aminopropyl) trimethoxysilane.

50. The method of claim 46, wherein said epoxy silane of said compostion is (3-glycidyloxypropyl) trimethoxysilane.

51. The method of claim 46, wherein said amine silane of said composition is (N,N-diethyl-3-aminopropyl) trimethoxysilane and said epoxy silane of said composition is (3-glycidyloxypropyl) trimethoxysilane.

52. The microarray of claim 51, wherein the concentration of said (N,N-diethyl-3-aminopropyl) trimethoxysilane ranges from about 0.01% to about 8% and the concentration of said (3-glycidyloxypropyl) trimethoxysilane ranges from about 0.01% to about 8%.

53. The method of claim 52, wherein the concentration of said (N,N-diethyl-3-aminopropyl) trimethoxysilane ranges from about 0.3% to about 4% and the concentration of said (3-glycidyloxypropyl) trimethoxysilane ranges from about 0.2% to about 4%.

54. The method of claim 53, wherein the concentration of said (N,N-diethyl-3-aminopropyl) trimethoxysilane is about 1% and the concentration of said (3-glycidyloxypropyl) trimethoxysilane is about 1%.

55. The method of claim 51, wherein the ratio of said (N,N-diethyl-3-aminopropyl) trimethoxysilane to said (3-glycidyloxypropyl) trimethoxysilane is about 1:1.

56. The method of claim 51, wherein the concentration of the combination of said (N,N-diethyl-3-aminopropyl) trimethoxysilane and said (3-glycidyloxypropyl) trimethoxysilane ranges from about 0.1% to about 1%.

57. The method of claim 56, wherein the concentration of the combination of said (N,N-diethyl-3-aminopropyl) trimethoxysilane and said (3-glycidyloxypropyl) trimethoxysilane ranges from about 0.15% to about 0.5%.

58. The method of claim 57, wherein the concentration of the combination of said (N,N-diethyl-3-aminopropyl) trimethoxysilane and said (3-glycidyloxypropyl) trimethoxysilane is about 0.2%.

59. The method of claim 46, further comprising tetraethylorthosilicate.

60. The method of claim 46, wherein said solid support comprises glasses, plastics and polymers.

61. A method of manufacturing a microarray for immobilization and hybridization of nucleic acid molecules, comprising the steps of:
a. immersing a solid support in a coating solution comprising an amine silane and an epoxy silane;
b. spinning said coated solid support in a spin coater to make a substrate;
c. spotting said nucleic acid molecules on the surface of said substrate; and
d. immobilizing said nucleic acid molecules on the surface of said substrate.

62. The method of claim 61, further comprising the step of incubating said substrate at an elevated temperature.

63. The method of claim 62, wherein said substrate is incubated at 80° C. for 20 hours.

64. The method of claim 61, wherein said nucleic acid molecules are immobilized on said substrate by placing said spotted substrate in a humid chamber.

65. The method of claim 64, wherein said humid chamber has a humidity of at least 60%.

66. The method of claim 64, wherein said nucleic acid molecules are immobilized at 42° C. for at least two hours.

67. The method of claim 61, wherein said nucleic acid molecules are immobilized on said substrate by UV irradiation.

68. The method of claim 61, wherein said nucleic acid molecules are oligonucleotides.

69. The method of claim 68, wherein said oligo-nucleotides range from 10-mer to 70-mer.

70. The method of claim 69, wherein said oligo-nucleotides range from 30-mer to 70-mer.

71. The method of claim 61, wherein said nucleic acid molecules are PCR products.

72. The method of claim 71, wherein said PCR products range from about 250 base pairs (bps) to about 2400 base pairs (bps).

73. The method of claim 72, wherein said PCR products range from about 980 bps to about 2300 bps.

74. The method of claim 61, wherein the concentration of said nucleic acid molecules ranges from about 0.2 to about 1.2 μg/μl.

75. The method of claim 68, wherein the concentration of said oligo-nucleic acids ranges from about 0.5 to about 1.2 μl.

76. The method of claim 71, wherein the concentration of said PCR products ranges from about 0.2 to about 0.5 μg/μl.

77. The method of claim 61, wherein said amine silane of said composition is (N,N-diethyl-3-aminopropyl) trimethoxysilane.

78. The method of claim 61, wherein said epoxy silane of said compostion is (3-glycidyloxypropyl) trimethoxysilane.

79. The method of claim 61, wherein said amine silane of said composition is (N,N-diethyl-3-aminopropyl) trimethoxysilane and said epoxy silane of said composition is (3-glycidyloxypropyl) trimethoxysilane.

80. The method of claim 79, wherein the concentration of said (N,N-diethyl-3-aminopropyl) trimethoxysilane ranges from about 0.01% to about 8% and the concentration of said (3-glycidyloxypropyl) trimethoxysilane ranges from about 0.01% to about 8%.

81. The method of claim 80, wherein the concentration of said (N,N-diethyl-3-aminopropyl) trimethoxysilane ranges from about 0.3% to about 4% and the concentration of said (3-glycidyloxypropyl) trimethoxysilane ranges from about 0.2% to about 4%.

82. The method of claim 81, wherein the concentration of said (N,N-diethyl-3-aminopropyl) trimethoxysilane is about 1% and the concentration of said (3-glycidyloxypropyl) trimethoxysilane is about 1%.

83. The method of claim 82, wherein the ratio of said (N,N-diethyl-3-aminopropyl) trimethoxysilane to said (3-glycidyloxypropyl) trimethoxysilane is about 1:1.

84. The method of claim 79, wherein the concentration of the combination of said (N,N-diethyl-3-aminopropyl) trimethoxysilane and said (3-glycidyloxypropyl) trimethoxysilane ranges from about 0.1% to about 1%.

85. The method of claim 84, wherein the concentration of the combination of said (N,N-diethyl-3-aminopropyl) trimethoxysilane and said (3-glycidyloxypropyl) trimethoxysilane ranges from about 0.15% to about 0.5%.

86. The method of claim 85, wherein the concentration of the combination of said (N,N-diethyl-3-aminopropyl) trimethoxysilane and said (3-glycidyloxypropyl) trimethoxysilane is about 0.2%.

87. The method of claim 79, further comprising tetraethylorthosilicate.

88. The method of claim 61, wherein said solid support comprises glasses, plastics and polymers.
